(19) United States
(12) Patent Application Publication
(10) Pub. No.: US 2015/0032092 A1
(43) Pub. Date: Jan. 29, 2015

(54) IMAGE-BASED COSMETIC SKIN TREATMENT SYSTEM

(71) Applicant: Yossef Ori Adanny, Mitzpe Ilan (IL)

(72) Inventor: Yossef Ori Adanny, Mitzpe Ilan (IL)

(21) Appl. No.: 14/337,117

(22) Filed: Jul. 21, 2014

Related U.S. Application Data
(60) Provisional application No. 61/858,679, filed on Jul. 26, 2013.
(60) Provisional application No. 61/858,682, filed on Jul. 26, 2013.

(51) Int. Cl. A61B 18/20

(52) U.S. Cl. CPC A61B 18/203; A61B 2018/1807
USPC 606/9

(57) ABSTRACT

There is provided an image-based system for cosmetic procedures for skin including an applicator including a light energy emitter, a camera operative to capture and communicate to a computer an image of a segment of skin or blemish obtained of the applicator being in contact with the skin and wherein the computer employs information extracted from the image to determine specific optimal treatment doses of at least one of light energy and RF energy for one or more skin fractions within the segment of skin. The system could also include a remote image capturing and processing device with at least one camera.

This is a utility patent application being filed in the United States as a non-provisional application for patent under Title 35 U.S.C. §100 et seq. and 37 C.F.R. §1.53(b) and claims the priority benefit of U.S. Provisional Application No. 61/858,679, filed 26 Jul. 2013 and U.S. Provisional Application No. 61/858,682, filed 26 Jul. 2013, both of which are incorporated herein by reference in their entirety.

### BACKGROUND

### Technical Field

The current method and apparatus relate to systems for cosmetic procedures for skin and in particular to image-based systems for cosmetic procedures for skin. External appearance is important to practically every person. In recent years, methods and apparatuses have been developed for various cosmetic procedures. These cosmetic procedures include wrinkle removal, scar removal, skin rejuvenation, skin resurfacing, hair removal, treatment of vascular lesions and others. In some of these cosmetic procedures, the skin and skin components are treated by one or more types of electromagnetic energy such as optical illumination (light) and radio frequency (RF).

When treating with light energy, the light may be monochromatic such as laser energy or polychromatic including a relatively narrow or broad spectrum of different wavelengths. The light energy depending on the wavelength may heat the skin and skin components such as hair and hair follicles to coagulate wounds, burn hair and destroy hair follicles, coagulate blood vessels in the follicles and produce photo-chemical effects. The time and intensity of the electromagnetic energy are selected to achieve a desired effect.

The light energy is applied to the skin and skin components employing an applicator having an aperture of a given dimension. The light energy is frequently applied in a pulse mode. Light energy applying devices achieve the desired effect only if a certain energy density is applied to the skin and skin components. Light energy treats the upper skin layer and penetrates to a relatively shallow depth of no more than few millimeters.

A typical cosmetic procedure for skin, such as skin resurfacing includes application of the light energy to blemishes having defined boundaries such as wrinkles, pigmented areas, acne scars, etc. the light energy is also applied to segments of skin adjacent to the blemish that do not necessarily require cosmetic treatment. A typical cosmetic hair removal procedure includes application of the light energy to a defined area of the skin. However, when using light energy for the cosmetic hair removal treatment the light energy is also applied to hairless areas adjacent to hair that do not necessarily require cosmetic hair removal treatment. These situations can result in unnecessary discomfort in the segments of skin being treated as well as in a waste of energy and increased wear of the machine. A solution to these disadvantages could be achieved by limiting the application of light energy only to the blemishes or hair and hair follicles and avoiding application of energy to other areas of skin.

Radio Frequency (RF) is applied to the skin employing two or more electrodes in contact with the skin. RF voltage is applied across the electrodes in pulse or continuous waveform (CW). The properties of the RF voltage are selected to generate RF induced current in a volume or layer of tissue to be treated. This current heats the skin tissue to the optimal temperature. For example, the temperature may bring about collagen structure changes or destruction, hair follicle destruction and other changes.

Professional equipment that combines light energy and RF energy treatment also exists. Usually this equipment is configured to illuminate a defined segment of a subject skin generally similar or equal to the surface of the aperture through which light energy is directed to the skin segment. The electrodes may heat deeper tissue layers than those heated by light energy so to reach, for example, deeper hair follicles.

There is a delicate balance between the amount of RF energy and light energy applied to the same skin segment. Exceeding the optimal proportion between them may lead to skin burns, whereas application of lower than optimal proportion RF energy and light energy may not bring the desired cosmetic results.

Additionally, skin is rarely uniform in appearance as a result of pigmentation, wrinkles, scars and other blemishes. Applying a uniform level of electromagnetic energy such as laser energy or laser energy combined with RF energy to a segment of skin including such blemishes or hair and hair follicles, may result in under heating some areas whereas overheating others. This is due to different energy (primarily light) absorption qualities of areas having various levels of pigmentation or differences in users skin types (i.e., having different levels of overall skin pigmentation).

A solution to this type of disadvantage could be achieved by limiting the application of light energy and RF energy only to the blemish or hair and hair follicles and avoiding application of energy to other segments of skin not necessarily requiring treatment as well as controlling the level of energy (light and/or RF) applied to each specific segment of skin.

Such a solution that provides selective treatment of skin fractions allows for higher energy levels to be applied to the skin fractions without scarring which accompanies treatment of larger areas of skin with the same energy level.

### SUMMARY

The current system and method seeks to provide an image-based system for cosmetic procedures for skin employing one or more types of electromagnetic energy selected from a group of types of electromagnetic energy including optical illumination (light) radio frequency (RF) energy, microwave energy and ultrasound energy.

There is thus provided in accordance with an example an applicator including a beamed light energy emitter such as a laser or, in some cases, IPL (Intense Pulse Light) or Light Emitting Diodes (LED) and a camera operative to communicate to a computer a captured image of a segment of skin including, for example, blemishes or hair. IPL can be used, for example, when a blemish is large enough, covering the full field of view thus negating the need for a narrow beam type of light energy such as that produced by a laser source of energy.

In accordance with another example, there is also provided an applicator including a plurality of discrete voltage-applying elements and a camera operative to communicate to a computer a captured image of a segment of skin or blemish or hair on the skin.

There is a delicate balance between the amount of RF energy and light energy applied to the same skin segment. Exceeding the optimal proportion between them may lead to skin burns, whereas application of lower than optimal proportion RF energy and light energy may not bring the desired cosmetic results. The applicator computer can employ information extracted from the image of the segment of skin captured by the camera to determine specific optimal treatment light energy or RF doses for one or more skin fractions within the segment of skin, blemishes or hair-containing skin segments.

The advantage of selective treatment of skin fractions rather than treatment of larger areas of skin is in that selective treatment of skin fractions allows for higher energy levels to be applied to the skin fractions resulting in less to no scarring of the treated skin, as would occur when treating larger areas of skin with the same energy level.

The system applicator is also operative to limit the application of light energy and RF energy to the blemish or hair or hair-containing fractions only and avoid application of energy to other segments of skin as well as controlling the level of energy (light and/or RF) applied to each specific segment of skin each smaller when in combination. This results in increased comfort in the segment of skin being treated as well as improved efficiency and decreased wear of the machine.

In accordance with yet another example, the computer can employ information extracted from at least one or more of the image of a segment of skin, manual input and sensors located on the applicator to formulate a cosmetic treatment protocol tailored to one or more of skin and blemish or hair parameters and to determine specific optimal treatment light energy doses for one or more blemish and/or hair-containing skin fractions within the segment of skin.

In accordance with still another example, the captured image could be displayed on a user interface touch-screen and allow a user employing a finger or a stylus to at least one or more of outline an area or a segment of skin within the displayed image to be treated or not treated, identify specific hairs or blemishes, scars or wrinkles to be treated and define a sequence and scanning pattern of application of light energy doses to two or more skin fractions within the segment of skin.

In accordance with still another example there is also provided a remote image capturing and processing device having a processor and a camera for capturing an image of hair on a segment of skin or blemish. The remote image capturing and processing device could communicate with a skin or blemish treatment device via a wired or wireless communication link.

In accordance with another example, there is also provided a method including obtaining or capturing an image of a segment of skin, hair or blemish, analyzing and processing the image and extracting from the image information regarding the hair and/or hair-containing segment of skin or blemish and determining specific optimal treatment doses of at least one or more of light energy and RF energy for one or more blemishes, hairs and/or hair-containing skin fractions within the segment of skin based on the extracted information.

### BRIEF DESCRIPTION OF THE DRAWINGS

The present method and system will be understood and appreciated more fully from the following detailed description, taken in conjunction with the drawings in which:

FIGS. 1A and 1B are simplified illustrations of an image-based system for cosmetic skin and hair removal procedures in accordance with two examples;

FIGS. 2A and 2B are simplified illustrations of patterns of application of light energy to fractions of skin in accordance with two examples;

FIGS. 3A and 3B are simplified illustrations of geometrical patterns formed by treated fractions of skin in accordance with an example;

FIGS. 4A and 4B are simplified illustration of an image of a skin segment captured by a camera of an image-based system for cosmetic skin procedures in accordance with an example;

FIGS. 5A and 5B are simplified illustration of a result of an analysis and processing of the image of FIG. 4;

FIGS. 6A and 6B are simplified illustration of a segment of skin in the image of FIG. 4 following light energy treatment;

FIGS. 7A, 7B, 7C, 7D, and 7E are simplified illustrations of an image-based system for cosmetic skin and hair removal procedures in accordance with another example;

FIGS. 8A and 8B are simplified illustration of an image of a skin segment captured by a camera of an image-based system for cosmetic skin and hair removal procedures in accordance with another example;

FIGS. 9A and 9B are simplified illustration of a result of an analysis and processing of the image of corresponding FIGS. 8A and 8B;

FIGS. 10A and 10B are simplified illustrations of an image-based system for cosmetic skin and hair removal procedures in accordance with yet two other examples;

FIGS. 11A and 11B are simplified illustration of segments of skin in the image of corresponding FIGS. 8A and 8B following RF treatment in accordance with an example;

FIG. 12 is a simplified illustration of an image-based system for cosmetic skin procedures in accordance with still another example;

FIG. 13 is a simplified illustration of a segment of skin in the image of FIG. 8A following RF treatment in accordance with another example;

FIG. 14 is a simplified illustration of an image-based system for cosmetic skin procedures in accordance with another example.

FIGS. 15A and 15B are simplified illustrations of an image-based system for cosmetic skin procedures in accordance with yet another example; and

FIG. 16 is a simplified illustration of an image-based system for cosmetic skin procedures in accordance with still another example.

### DETAILED DESCRIPTION

The term “Hair Removal” as used in the below disclosure means removal or destruction of a hair shaft and damaging or destruction of the hair follicle.

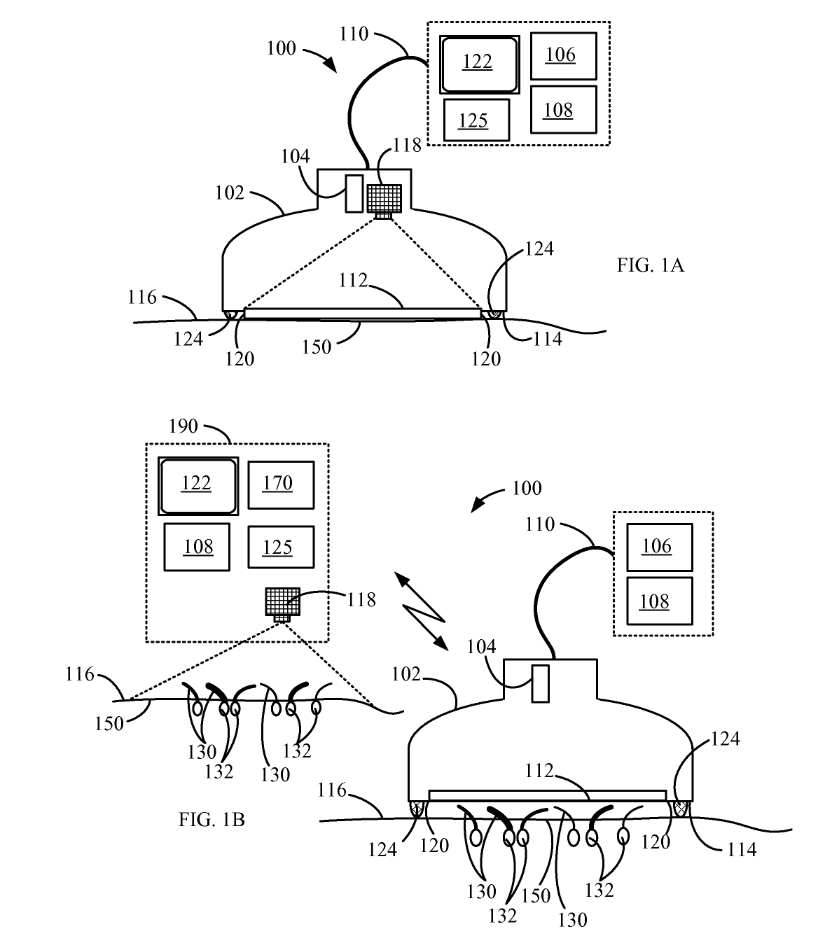

Referring now to FIG. 1A, which is a simplified cross-section view and block illustration of an image-based system for cosmetic procedures for skin in accordance with an example. System 100 for cosmetic skin procedures could include an applicator 102 including a light energy emitter 104 communicating via a harness 110 with a source of light energy 106 and a computer 108.

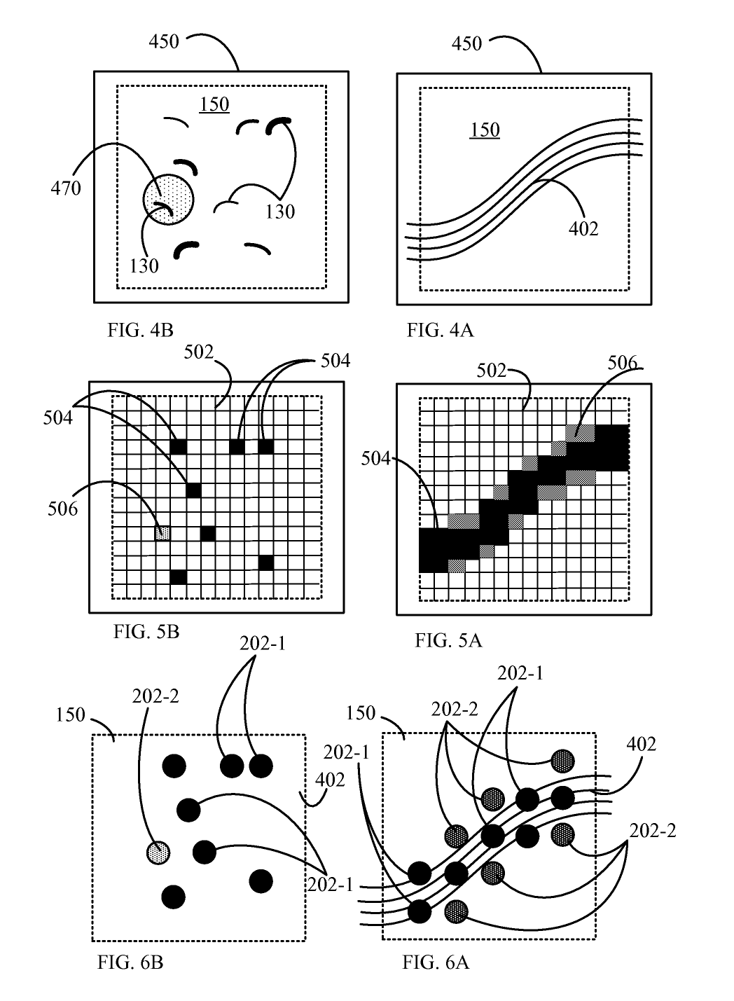

Light energy emitter 104 can apply light energy to a segment 150 of skin 116 through an aperture 112 in a surface 114 of applicator 102 facing and optionally being in contact with skin 116 during a cosmetic procedure session. Aperture 112, as used in this disclosure is defined by an opening that could be but not necessarily be covered by a transparent rigid or semi-rigid surface such as plastic, glass or similar. Applicator 102 could also include a camera 118, such as, for example, a digital CCD camera, a CMOS camera or similar, communicating with computer 108 via harness 110 or via standard wireless communication links such as Bluetooth or similar and operative to communicate to computer 108 images, such as image 450 (FIG. 4) of a segment 150 of skin 116 captured through aperture 112 as depicted by phantom lines (FIGS. 1A and 1B).

The area of segment 150 could be the same size, larger or smaller than an area defined by borders 120 (FIGS. 1A and 1B) of aperture 112. System 100 could also include a display 122 operative to display in real time images captured by camera 118 or images stored and retrieved for comparison purposes from an image bank in computer 108 memory. Camera 118 could provide an image of the segment of skin.

Additionally and optionally, system 100 could automatically scan a large segment of skin (e.g., a face of a user) and communicate images of the scan captured by camera 118 to computer 108, which could display on display 122 images of the scanned segment of skin to be treated or not treated, identify to system 100 specific blemishes, scars or wrinkles or hair to be treated and define a sequence and scanning pattern of application of light energy doses to the skin fractions within the scanned area of skin as will be explained in greater detail below. Computer 108 could also display a list of blemishes or areas with hair to be treated and have the user confirm the listed blemishes or areas with hair to be treated. Once confirmed, computer 108 could automatically activate application of appropriate light energy doses to confirmed blemishes to be treated.

Optionally, applicator 102 could also include a user interface 125 to provide for manual input by a system 100 operator. Additionally and optionally, user interface 125 could include a touch-screen, which could be display 122 or an additional display operative to display an image captured by camera 118 and allow a user, employing a finger or a stylus to outline an area or a segment of skin 150 within the displayed image to be treated or not treated, define a sequence of light energy or RF energy application, identify to system 100 specific hairs 130, blemishes, scars or wrinkles to be treated and define a sequence and scanning pattern of application of light energy doses to two or more skin fractions within the segment of skin.

Alternatively and optionally, and as shown in FIG. 1B, camera 118, display 122, interface 125 and a remotely placed computer 108, could be included in a remote image capturing and processing device 190 having a wired or wireless communication link with applicator 102 computer 108. Device 190 could be operative to capture an image such as, for example, image 450 (FIG. 4) of hair on skin 1164) and process the captured image, generate an analysis, e.g., in a form of a mapped X-Y grid 502 (FIG. 5), determine and communicate specific optimal treatment doses of light energy and/or RF energy for one or more fractions 202 within segment 150 of skin 116 and communicate the specific optimal treatment doses to computer 108.

Device 190 processor 140 could either store the generated analysis and/or communicate the generated analysis to applicator 102 computer 108. Alternatively and optionally, device 190 processor 140 could analyze and process an image captured by camera 118, and communicate to applicator 102 information regarding specific optimal treatment light energy doses and application pattern for one or more skin fractions 202 within segment 150 of skin 116 based on the generated analysis. This reduces the cost of the handheld applicator.

Alternatively and optionally, device 190 computer 108 could either store the raw image and/or communicate the raw captured image to applicator 102. Computer 108 could analyze and process by the image as it will be explained in greater detail below.

Additionally and optionally, device 190 could store raw captured image and/or the generated analysis or communicate to applicator 102 the location of the captured image on the body of a user as well as other information extracted from the image.

Additionally and optionally, when functioning as a hair removal system, system 100 device 190 could store raw captured image and/or the generated analysis or communicate to applicator 102 not just the location of the captured image on the body of a user but other information as well regarding hair parameters selected from a group of parameters including number of hairs 130, density of hair 130, pigment of hair 130, length and thickness of hair 130 and location of hairs 130 within segment 150.

The communicated information from device 190 could be stored by applicator 102 in a memory for use during a future cosmetic procedure session or for cosmetic procedure session statistics in a situation in which the communicated information is post-treatment information.

In this embodiment, removal of camera 118 could render applicator 102 to be lightweight and smaller and easier to manipulate by an operator. Additionally, applicator 102 could become a handheld autonomous unit communicating with device 190 via wire or wireless communication link.

Optionally, applicator 102 could also include sensors 124 selected from a group of sensors including temperature sensors, contact sensors, and impedance sensing mechanism that could be located in the applicator or in computer 108.

Light energy emitter 104 could be any form of light energy applied in a light beam form such as a laser selected from a group of lasers including gas lasers, solid-state lasers, fiber lasers, semiconductor lasers, dye lasers and similar, e.g., a Alexandrite, Nd:Yag, CO₂ laser, ER:YAG laser, laser diodes as well as non-coherent light such as Intense Pulse Light (IPL) sources and Light Emitting Diodes (LED). IPL can be used, for example, when a blemish is large enough, covering the full field of view thus negating the need for a narrow beam type of light energy such as that produced by a laser source of energy.

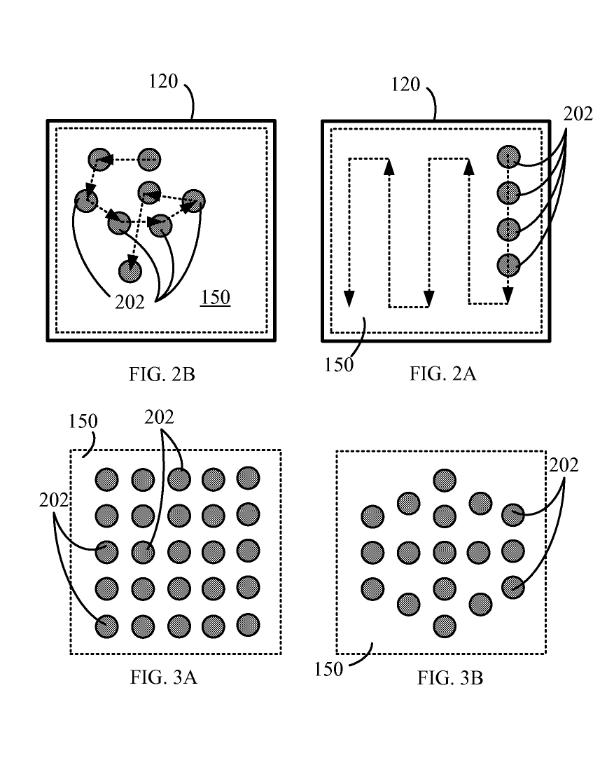

During a cosmetic procedure session applicator 102 could be coupled to segment 150 of skin 116 at a desired location on a subject's body and light energy could be applied to skin 116 via aperture 112. The light energy could be applied to skin 116 in a fractional manner, applying pulses of energy to fractions 202 (FIG. 2) of skin in a stepwise (e.g., pulsed) vector scanning fashion in a predetermined pattern an example of which is illustrated in FIG. 2. Each pulse could be applied to a single fraction of skin 202 having a radius between 20 and 1000 micron thus treating between 1 and 300 fractions of skin within 1 cm². Alternatively and optionally, when functioning as a hair removal system, the light energy could be applied to specific single hairs 130 or hair follicles 132 region by applying the light beam to the base of hair 130 such as to penetrated the skin and destroy the follicle.

The sequence of light pulse application as well as the scan pattern and the final pattern of treated fractions of skin 202 could vary in accordance with, for example, the type of cosmetic procedure performed, the area of the subject's body on which the cosmetic procedure is to be performed, the type of skin being treated, pigmentation of hair 130 being treated, the thickness of hair 130 being treated, etc. Additionally and optionally, in hair treatment procedures, computer 108 could also employ information extracted from an image 450 (FIG. 4) to determine specific individual hair treatment light energy and optimal light energy scanning patterns.

FIGS. 2A and 2B, collectively referred to as FIG. 2, illustrate sequence patterns of application of light pulses to a segment 150 of skin 116 by light energy emitter 104 in accordance with two examples. In FIG. 2A, light energy is applied in a stepwise (e.g., pulsed) serpentine-like scanning pattern treating fractions 202 of skin 116. In FIG. 2B, light energy is applied in another stepwise (e.g., pulsed) vector scanning pattern treating fractions 202 of skin 116. It would be appreciated by those skilled in the art that any suitable light energy dose application sequence and/or pattern to skin 116 segment 150 could be generated by computer 108 including, but not limited to, a random sequence and/or pattern of light energy dose application.

FIGS. 3A and 3B, collectively referred to as FIG. 3, depict geometric patterns of treated fractions 202 of skin 116 following treatment with light energy applied to skin 116 by light energy applying emitter 104. In FIG. 3A treated fractions 202 of skin 116 form a square geometric pattern wherein in FIG. 3B fractions 202 of skin 116 form a hexagonal geometric pattern.

Reference is now made to FIGS. 4A and 4B collectively referred to as FIG. 4, which are images of a skin segment captured by camera 118 of system 100 applicator 102 (FIG. 1A) or device 190 (FIG. 1B) in accordance with an example.

FIG. 4A depicts an image 450 of a blemish 402 in skin 116 segment 150 captured by camera 118 through aperture 112. Blemish 402 could be, for example, a wrinkle, a scar, a pigmented area or similar. The segment of skin 150 captured by camera 118 is defined by borders 120 of aperture 112 and could be the same size, smaller or larger than segment 150 of skin 116. As shown in FIG. 4, segment 150 is smaller than the image captured by camera 118. FIG. 4B depicts an image 450 of hair 130 on skin 116 segment 150 captured by camera 118 through aperture 112. The area of skin captured by camera 118 could be defined by borders 120 of aperture 112 and could be the same size or larger than segment 150 of skin 116.

Image 450 could be communicated to computer 108 and could be stored in computer 108 memory. Additionally or alternatively, image 450 could be analyzed and processed by computer 108 to extract information regarding hair and/or skin parameters in general. One or more blemish parameters could include, for example, skin pigmentation, blemish 402 thickness, for example in cases in which blemish 402 includes scar tissue could be extrapolated from its width employing a lookup table and blemish depth, for example in cases in which blemish 402 is a wrinkle, a depth thereof could be measured from a 3D image. Hair 130 parameters can be parameters selected from a group of parameters including number of hairs 130, pigment of hair 130, length and thickness of hair 130, location of hairs 130 within segment 150, thickness of skin 116 or depth of follicles 132, skin pigmentation, etc.

Additionally and optionally, a visible light outline such as a visible light laser beam could be used to enable a user to visualize area or segment 150 to be treated. Alternatively and optionally, the visible light could outline for system 100 a desired area or segment 150 to be treated and/or to be captured by camera 118 for analysis and processing by computer 108 so that to receive a cosmetic treatment protocol formulated by computer 108 and tailored to the selected area or segment 150 captured by camera 118. Alternatively and optionally, the captured image of the desired segment 150 could be stored in computer 108 memory for future treatment or reference.

As depicted in FIG. 4B, hairs 130 distributed over a segment 150 of skin 116 could be of various lengths and widths as well as degree of pigmentation. Computer 108 and processor 140 could employ information extracted from one or more of image 450, manual input and sensors 124 to formulate a cosmetic hair 130 removal protocol tailored to particular hair 130, skin 116 and/or blemish 402 parameters and to determine specific optimal treatment light energy doses for one or more skin fractions 202 within segment 150 of skin 116 based on the extracted information.

Additionally and optionally, computer 108 could also employ information extracted from image 450 to determine specific light energy scanning patterns. The extracted information could include one or more skin and blemish parameters including skin type, location of the segment of skin on the body, level of pigmentation, type of blemish, temperature and/or impedance of segment of skin, level of skin hydration, blemish thickness and/or depth and blemish shape and location within the skin segment as well as one or more hair 130 parameters.

Additionally and/or optionally, system 100 could also use image 450 to monitor cosmetic procedure progress by comparing a currently captured image of the segment 150 of skin or blemish with an image of hair 130 on a segment of skin 150 or blemish 402 captured at a previous treatment or prior to treatment and stored in a memory of the skin segment.

Skin 116 segment 150 could also include an area 470 including a hair 130 or blemish 402 to be removed, which could be a pigmented or a hypo-pigmented (blanched) area of skin which would require a dose of light energy different than doses of light energy required in areas surrounding area 470 within skin 116 segment 150 remote image capturing and processing device.

Reference is now made to FIGS. 5A and 5B collectively referred to as FIG. 5, which are simplified illustrations of a result generated by computer 108 of an analysis and processing of the images of FIG. 4. Computer 108 could extract from image 450 information regarding hair 130 or blemish 402 location on skin 116 segment 150, analyze and process the extracted information in combination with other parameters such as the hair, blemish and skin parameters described above and input into system 100 computer 108 and convert into optimal light energy dose values to be applied to each individual hair 130 and/or hair-containing fraction 202 or blemish 402 of skin 116 as well as the light energy scanning pattern at which the light energy is to be applied to segment 150 of skin 116.

Optimal light energy dose values for the treatment of hair on skin 116 segment 150 and/or blemish 402 could be, for example, mapped on an X-Y grid 502. Grid 502 could be, for example, divided into squares the size of which corresponds to the size of fractions 202 (FIGS. 2 and 3) of skin 116 (FIGS. 1A and 1B). As depicted in FIG. 5, determination of the dose of light energy to be applied at each grid square could be based on skin 116 parameters such as skin type, location of segment 150 on the body, level of hair 130 pigmentation, type of hair 130 or blemish 402, skin 116 segment 150 temperature and/or impedance obtained from sensors 124, level of skin 116 (FIGS. 1A and 1B) hydration, hair 130 location within the skin segment, blemish 402 thickness and/or depth, blemish shape and location within the skin segment some of which could be obtained from image 450 while others could be input manually by system 100 (FIGS. 1A and 1B) operator. The light energy dose could also depend on a user skin type and relative difference in pigmentation between the blemish or hair pigmentation and the pigmentation of the users skin type.

Additionally, the dose of light energy could also depend on the type of light energy, the pulse frequency and duration as well as optimal scanning/energy application scanning pattern and other machine factors.

As shown in FIG. 5, areas within segment of skin 150 requiring, for example, a higher dose 504 of light energy are marked on grid 502 in black, whereas areas within segment of skin 150 requiring, for example, a lower dose 506 of light energy are marked on grid 502 in grey. Computer 108 could convert the result shown in FIG. 5 into a treatment protocol in accordance with a cosmetic procedure to be carried out on skin 116 segment 150.

The protocol could be stored in computer 108 memory for use at a later time, to compare treatment parameters to prior performed treatments stored in computer 108 memory to enable a user to track progress of treatment results or for repeated use in cases of multiple treatments, displayed on display 122, printed out on a printer (not shown), communicated to a remote computer by a wired or wireless communication or automatically control the light energy dose applied to various fractions 202 of skin 116 in accordance with a predetermined protocol defining preset light energy dose levels corresponding to an obtained set of image and/or hair, blemish or skin parameters.

FIGS. 6A and 6B, collectively referred to as FIG. 6, are simplified illustration of a segment 150 of skin 116 captured in image 450 of FIG. 4 following light energy treatment, demonstrates application of light energy to a segment 150 of skin 116 (FIGS. 1A and 1B) in accordance with an example. Light energy is applied to skin 116 segment 150 based on the mapping shown in FIG. 5 Skin 116 fractions 202-1 corresponding to areas requiring, for example, a higher dose 504 of light energy marked on grid 502 (FIG. 5) are shown to have received a high dose of light energy and are marked in black, whereas skin 116 hair-containing fractions 202-2, corresponding to areas within segment of skin 150 requiring, for example, a lower dose 506 of light energy on grid 502 (FIG. 5) are shown to have received a low dose of light energy and are marked in grey. Other (e.g, hairless or blemishless) areas within segment 150 may or may not receive a dose of light energy in accordance with a cosmetic procedure protocol derived as explained above.

At present, when using light energy for the cosmetic treatment of distinct hairs or blemishes having defined boundaries such as wrinkles, pigmented areas, acne scars, etc. the light energy is also applied to hairless or blemishless areas within segment of skin 150 adjacent to the hair or blemish that do not necessarily require cosmetic treatment.

A solution to these disadvantages could be achieved by limiting the application of light energy to only hairs 130 or blemishes 402 based on a captured image 450 of a segment 150 of skin 116 to be treated and avoiding application of energy to other hairless or blemishless areas within segment of skin 150 that do not necessarily require cosmetic treatment, thus minimizes unnecessary discomfort in the area of skin being treated as well as a waste of energy and increased wear of system 100.

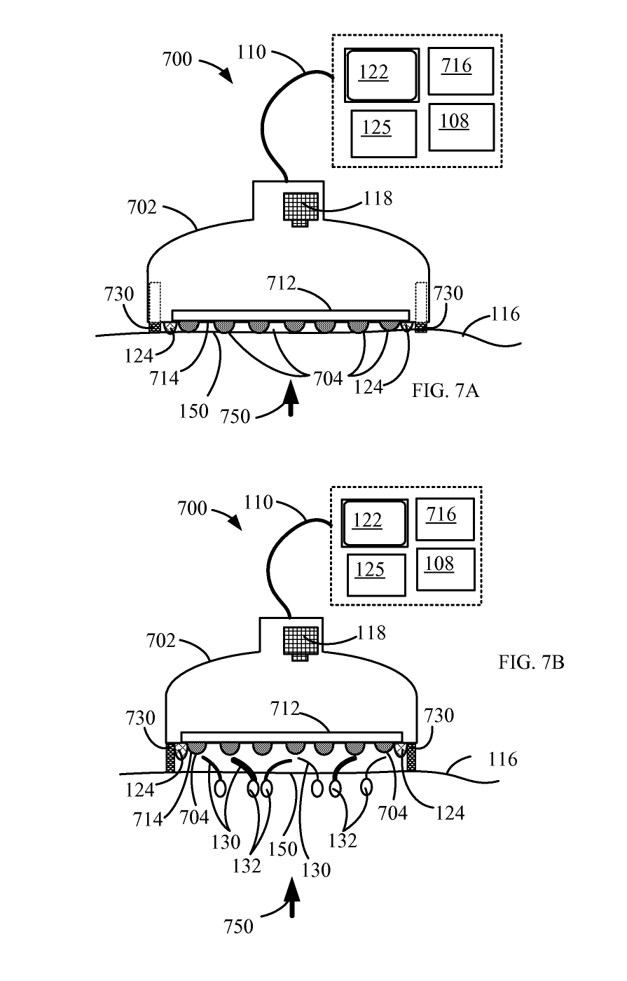

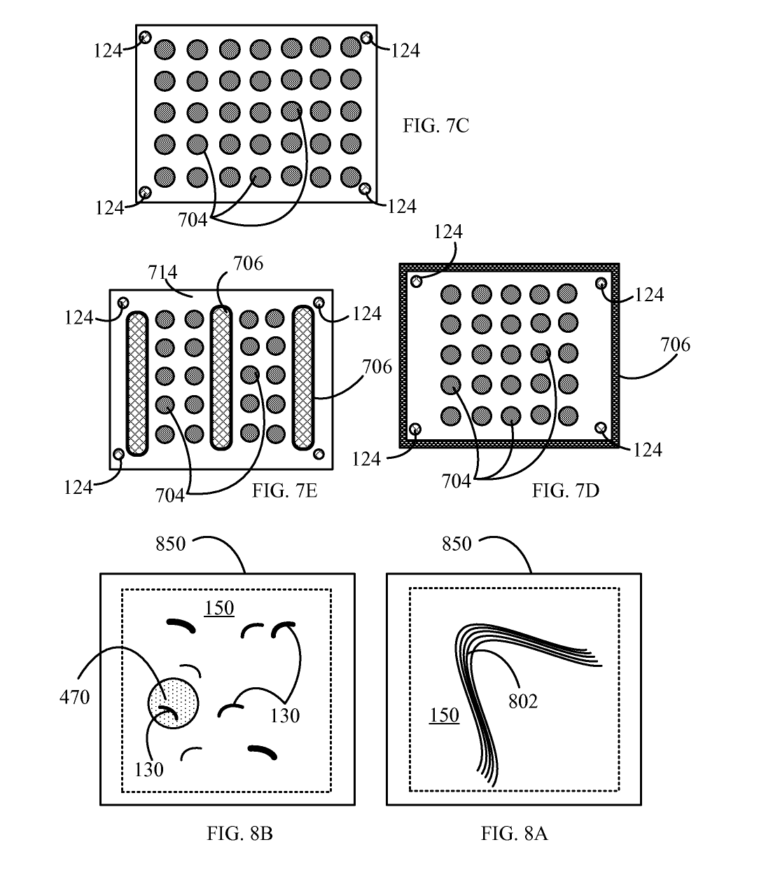

Reference is now made to FIGS. 7A, 7B, 7C, 7D and 7E, which are simplified illustrations of an image-based system for cosmetic procedures for skin in accordance with another example. FIGS. 7C and 7D are views of an applicator 702 of FIGS. 7A and 7B as viewed from a direction indicated by arrow 750. System 700 applicator 702 could include a plurality of discrete voltage-applying elements 704 formed on surface 714, and optionally around, aperture 712 in surface 714 of applicator 702 being in contact, via discrete voltage-applying elements 704 with skin 116 during a cosmetic procedure session. Discrete voltage-applying elements 704 could be in communication with a source of RF electrical energy 716 and computer 108. FIG. 7E depicts another example of an arrangement of a plurality of discrete voltage-applying elements 704 and ground electrodes 706 on surface 714 of applicator 702.

Applicator 702 could also include a camera 118 such as that described above and, optionally, a display 122 and user interface 125 to provide for manual input by system 100 operator.

Additionally and optionally, when functioning in hair removal cosmetic procedures, system 100, applicator 702 depicted in FIG. 7B could also include reversibly extendable spacers 730, operative to reversibly extend from applicator 702 surface 714, to allow hairs 130 to remain erect while camera 118 obtains an image such as image 450 (FIG. 4) of skin 116 segment 150.

Discrete voltage-applying elements 704 could be individually controlled and activated by computer 108 and couple voltage such as RF energy to hairs 130 and follicles 132 or blemishes in fractions 202 (FIG. 2) of skin 116 directly below or therebetween. Discrete voltage-applying elements 704 could be arranged in rows such as shown in FIG. 7C or in any other appropriate geometrical pattern. Applicator 702 discrete voltage-applying elements 704 could be activated in asymmetric bipolar mode or monopolar mode in which case surface 714 of the tip of applicator 702 may also include a return or ground electrode such as, for example, electrode 706 (FIGS. 7D and 7E) located along the periphery of applicator 702 surface 714 or a number of ground electrodes 706 located (not shown) in contact with skin elsewhere on the subject's body.

FIGS. 8A and 8B, collectively referred to as FIG. 8, which are simplified illustrations of an image of a skin 116 segment 150 captured by a camera 118 of an image-guided system 100 for cosmetic procedures for skin in accordance with another example, shows an image 850 of a segment 150 of skin 116 including a blemish 802 (FIG. 8A) and hair 130 and a pigmented area 470 (FIG. 8B) such as, for example, a hyper-pigmented or a hypo-pigmented (blanched) area of skin captured by camera 118 (FIG. 7A). Blemish 802 could be, for example, a wrinkle, a scar, a pigmented area or similar. The segment of skin captured by camera 118 is defined by borders 120 of aperture 112 and as shown in FIG. 4, could be the same size, smaller or larger than segment 150 of skin 116.

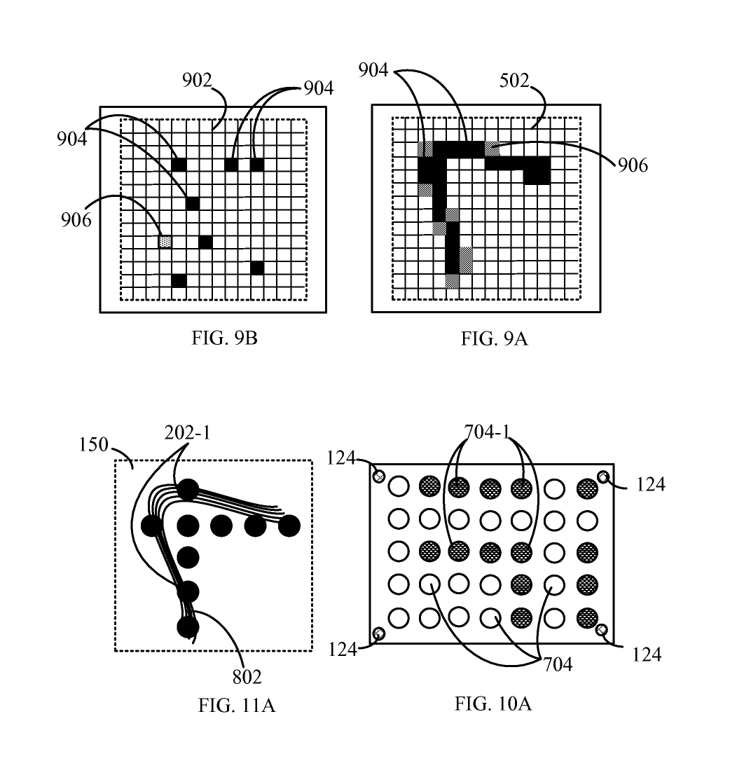

FIGS. 9A and 9B, collectively referred to as FIG. 9, which are simplified illustrations of a result of an analysis and processing of the corresponding images of FIG. 8, illustrate an example of results of computer 108 analysis and processing of an image 850 as described above and determination of the optimal dose of RF energy to be applied at each grid square based on hair 130, blemish 802 and skin 116 parameters. Such parameters could include skin type, location of segment 150 on the body, skin 116 segment 150 temperature and/or impedance obtained either from sensors 124 or from discrete voltage-applying elements 704, level of skin 116 (FIG. 1) hydration, number of hairs 130, pigment of hair 130, length and location of hair 130 within the skin segment 150, type of blemish 802 and/or depth, blemish shape and others, some of which could be obtained from a captured image 450 while others could be input manually by system 100 (FIG. 1) operator.

Additionally, the dose of RF energy could also depend on the pulse frequency and length as well as voltage, skin impedance and other machine factors.

As shown in FIG. 9 and as described above, areas within segment of skin 150 requiring a higher dose of RF energy 904 are marked on grid 502 in black, whereas areas within segment of skin 150 requiring a lower dose of RF energy 906 are marked on grid 502 in grey.

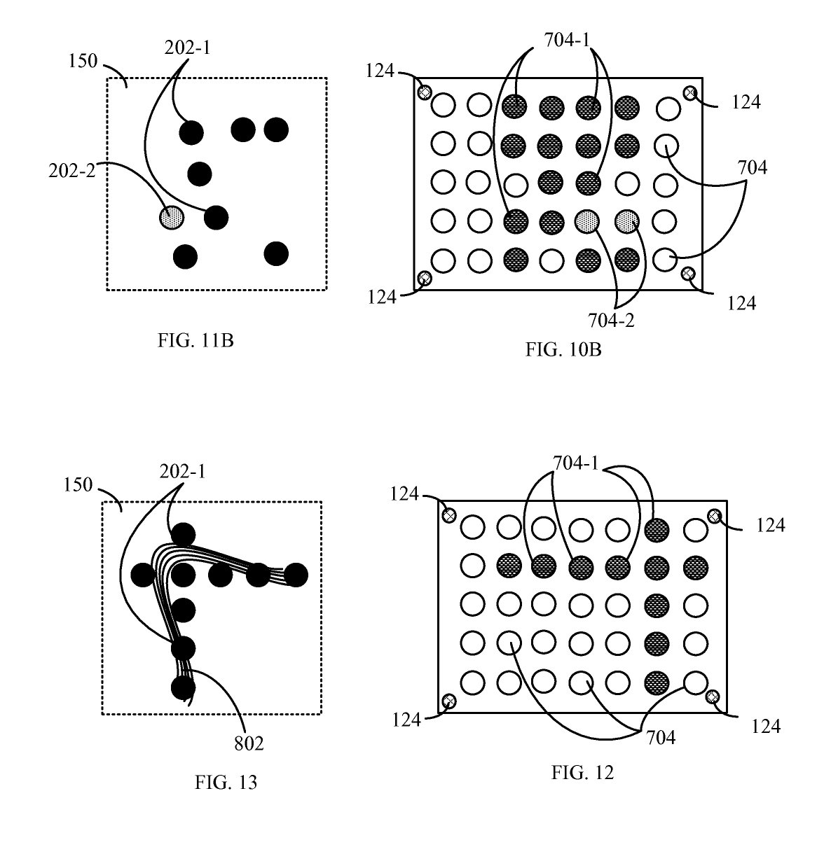

FIG. 10A, which is a simplified illustration of an image-based system for cosmetic procedures for skin in accordance with another example, illustrates a view of applicator 702 of FIG. 7A as viewed from a direction indicated by arrow 750 and depicts discrete voltage-applying elements 704 activated by computer 108 in a bipolar configuration in accordance with the captured image 850 of FIG. 8A. Elements 704-1 are shown to apply a higher dose of RF energy. Remaining elements 704 are shown to be in an Off mode and apply no RF energy at all. The applied optimal treatment RF energy doses for blemish 802 result from information extracted from an analysis and processing of image 850 of FIG. 8A captured by camera 118 and carried out by computer 108.

As described above, computer 108 could convert the result shown in FIG. 9 into a treatment protocol in accordance with a cosmetic procedure to be carried out on skin 116 segment 150. The protocol could be stored in computer 108 memory for use at a later time or repeated use e.g., as a basis for comparison, in cases of multiple treatments, displayed on display 122, printed out on a printer (not shown), communicated to a remote computer by a wired or wireless communication or automatically control the RF dose applied to various fractions 202 of skin 116 in accordance with a predetermined protocol defining preset RF energy dose levels corresponding to an obtained set of image and/or skin parameters.

FIG. 11A, which is a simplified illustration of a segment of skin in the image of FIG. 8A following RF treatment in accordance with an example, demonstrates application of RF energy to a segment 150 of skin 116 (FIG. 1) in accordance with another example. Based on the mapping shown in FIG. 10A, RF energy could be applied to skin 116 fractions 202-1 corresponding to areas within segment of skin 150 requiring a higher dose of RF energy 904 marked on grid 502 (FIG. 9A). In FIG. 11A, fractions 202-1 are shown to have received, for example, a dose of RF energy and are marked in black. Other non-treated areas within segment 150 are shown to have not received any dose of RF energy in accordance with a predetermined cosmetic procedure protocol.

FIG. 10B illustrates a view of applicator 702 of FIG. 7B as viewed from a direction indicated by arrow 750 and depicts discrete voltage-applying elements 704 activated by computer 108 in a asymmetric bipolar configuration in accordance with the captured image 850. Elements 704-1 are shown to apply a higher dose of RF energy whereas elements 704-2 are shown to apply a lower dose of RF energy. Remaining elements 704 are shown to be in an Off mode and apply no RF energy at all. The applied optimal treatment RF energy doses for hair 130 result from information extracted from an analysis and processing of image 850 captured by camera 118 and carried out by computer 108.

Computer 108 could activate elements 704-1, appearing in dark grey (high dose) in FIG. 10B and elements 704-2, appearing in light grey (low dose) in FIG. 10B, based on the extracted information. The remaining discrete voltage-applying elements 704 are not activated (in the Off mode) and appear in white.

FIG. 11B demonstrates application of RF energy to hairs 130 of a segment 150 of skin 116 (FIG. 1B) in accordance with another example. Based on the mapping shown in FIG. 10B, RF energy could be applied to hairs 130 of skin 116 fractions 202-1 corresponding to areas requiring a higher dose of RF energy 904 marked on grid 502 (FIG. 10B). In FIG. 11B, fractions 202-1 are shown to have received, for example, a higher dose of RF energy and are marked in black, whereas fractions 202-2 have received, for example, a lower dose of RF energy and are marked in grey. Other hairless areas of segment 150 are shown to have not received any dose of RF energy in accordance with a predetermined cosmetic procedure protocol.

FIG. 12, which is a simplified illustrations of an image-based system for cosmetic procedures for skin in accordance with another example illustrates a view of applicator 702 of FIG. 7A as viewed from a direction indicated by arrow 750 and depicts discrete voltage-applying elements 704-1 activated by computer 108 in a monopolar configuration. The applied optimal treatment energy doses for blemish 802 result from information extracted from an analysis and processing of image 850 captured by camera 118 carried out by computer 108.

Computer 108 could activate elements 704-1 appearing in grey in FIG. 12, based on the extracted information. The remaining discrete voltage-applying elements 704 are not activated (in the Off mode) and appear in white. In this configuration, a return electrode 706 could be located, for example and as shown in FIG. 7D, along the periphery of applicator 702 surface 714 or in contact with skin elsewhere on the subject's body.

FIG. 13, which is a simplified illustration of a segment of skin in the image of FIG. 8A following RF treatment in accordance with another example demonstrates application of RF energy to a segment 150 of skin 116 (FIG. 1A). Based on the mapping shown in FIG. 9A, RF energy could be applied to skin 116 fractions 202-1 corresponding to areas within segment of skin 150 requiring a higher dose of RF energy 904 marked on grid 502 (FIG. 9A). In FIG. 13, fractions 202-1 are shown to have received a higher dose of RF energy and are marked in black. Other areas within segment of skin 150 may or may not be receive a dose of RF energy in accordance with a predetermined cosmetic procedure protocol.

When employing an applicator such as applicator 702, image-based system 100 for cosmetic procedures for skin employs information extracted by computer 108 from image 850 to formulate a cosmetic treatment protocol tailored to the specific skin 116, hair 130 or blemish 802 parameters derived from image 850 (FIG. 8) and to determine specific optimal treatment energy doses for each skin 116 fraction 202 within segment 150 of skin 116.

This limits the application of RF energy only to hairs 130 or blemish 802 based on a captured image 850 of a segment 150 of skin 116 to be treated avoiding application of energy to other areas of skin 116 within segment 150 that do not necessarily require cosmetic treatment and thus minimizes unnecessary discomfort in the segment of skin 150 being treated as well as a waste of energy and increased wear of system 100.

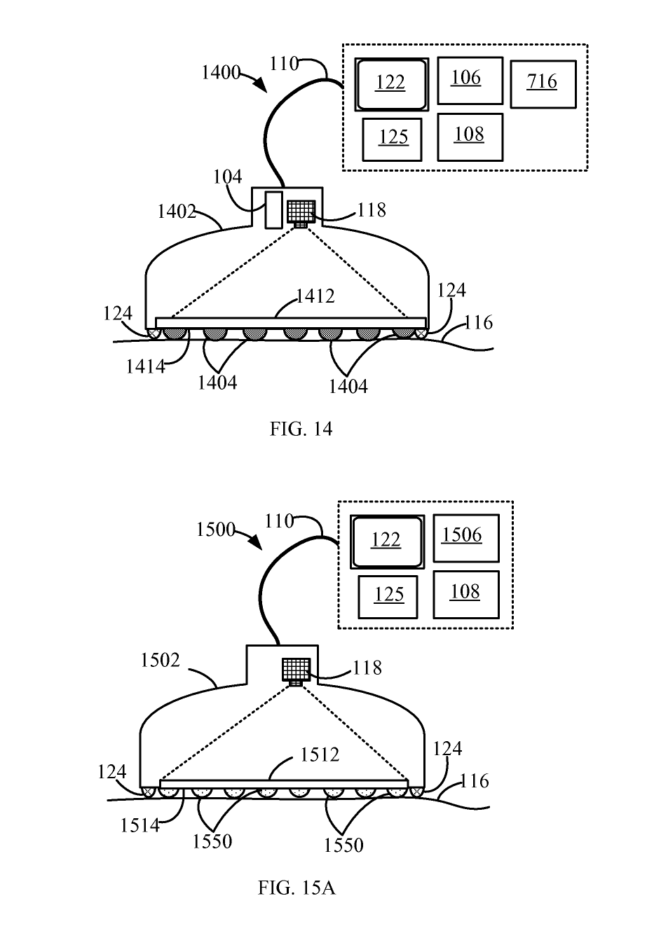

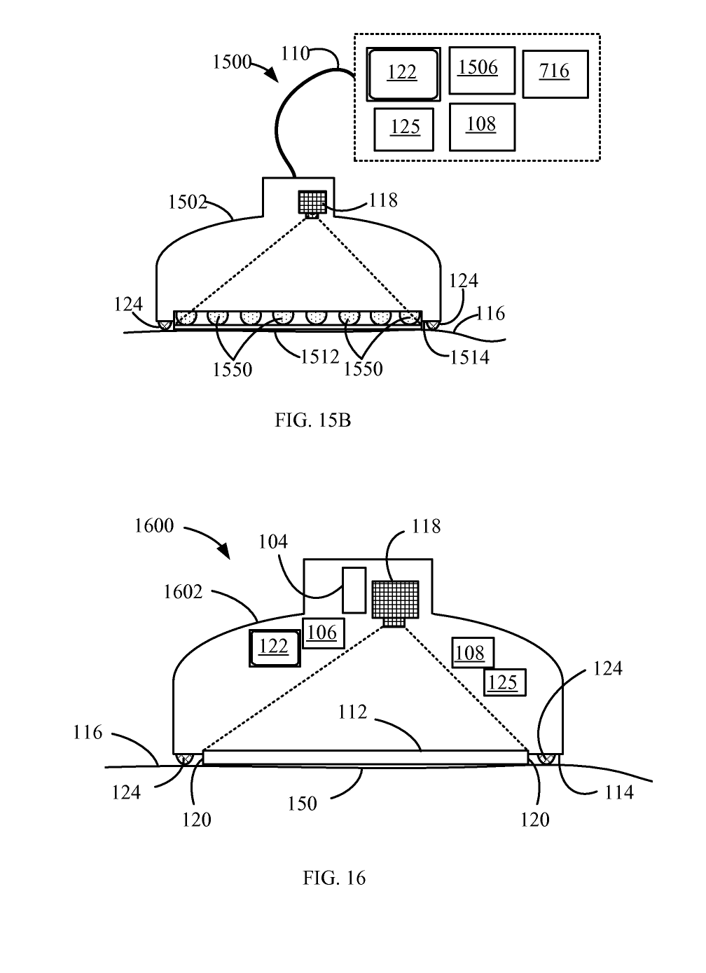

Reference is now made to FIG. 14, which is a simplified illustration of an image-based system for cosmetic procedures for skin in accordance with another example. System 1400 could include an applicator 1402 housing a light energy emitter 104 communicating with a source of light energy 106 and a computer 108 via a harness 110. Light energy emitter 104 can apply light energy to a segment of skin through an aperture 1412 in a surface 1414 of applicator 1402 facing hairs 130 and/or being in contact with skin 116 during a cosmetic procedure session.

Applicator 1402 could also include a camera 118 communicating with computer 108 via harness 110 or via wireless communication such as BlueTooth™ or similar and operative to communicate to computer 108 images of a segment 150 of skin 116 captured via aperture 1412 as depicted by phantom lines. The area of segment 150 could be the same size, larger or smaller than an area defined by borders 120 of aperture 1412. System 1400 applicator 1402 could also include a plurality of discrete voltage-applying elements 1404, similar to the discrete voltage-applying elements described in FIGS. 7A and 7B, protruding from, and optionally around, aperture 1412 in surface 1414 of applicator 1402 being in contact, via elements 1404 with skin 116 during a cosmetic procedure session.

Elements 1404 could be in communication with a source of RF electrical energy 716 and computer 108. System 100 could also include a display 122 operative to display in real time images captured by camera 118 or images stored and retrieved from an image bank in computer 108 memory. Camera 118 could be a digital camera such as a CCD or CMOS camera.

Optionally, applicator 1402 could also include a user interface 125 to provide for manual input by a system 1400 operator. Optionally, applicator 1402 could also include sensors 124 selected from a group of sensors including temperature sensors, impedance sensors and contact sensors.

There is a delicate balance between the amount of RF energy and light energy applied to the same skin segment. Exceeding the optimal proportion between them may lead to skin burns, whereas application of lower than optimal proportion RF energy and light energy may not bring the desired cosmetic results.

Additionally, skin is rarely uniform in appearance as a result of pigmentation, wrinkles, scars and other blemishes. Applying a uniform level of electromagnetic energy such as laser energy, RF energy or a combination of both to an segment of skin 150 including such blemishes may result in under heating some areas within segment of skin 150 whereas overheating others. This is due to different energy (primarily light) absorption qualities of the various blemishes. A solution to this type of disadvantage and as described above could be achieved by limiting the application of light energy to the hair or blemish and avoiding application of energy to other hairless or blemishless areas within segment of skin 150 as well as controlling the level of energy (light and/or RF) applied to each specific fraction 202 of segment 150 of skin 116.

Also, when employing an applicator such as applicator 1402 of image-based system 1400 for cosmetic procedures for skin, computer 108 could employ information extracted from one or more of images 450 (FIG. 4) and 850 (FIG. 8), manual input and sensors 124 to formulate a cosmetic treatment protocol tailored to skin 116, hair 130 and/or a blemish 402 (FIG. 4)/802 (FIG. 8) parameters and to determine specific optimal treatment light energy doses for one or more skin fractions 202 within segment 150 of skin 116 based on the extracted information.

Reference is now made to FIGS. 15A and 15B collectively referred to as FIG. 15, which are simplified illustrations of an image-based system for cosmetic procedures for skin in accordance with other examples. Applicator 1500 could include one or more Light Emitting Diodes (LED) 1550 arranged in a similar fashion to the discrete voltage-applying elements 704 described in FIGS. 7A and 7B. As shown in FIG. 15A, LEDs 1550 could be protruding from, and optionally around, aperture 1512 in surface 1514 of applicator 1502 being in contact, via LEDs 1550, with hairs 130, blemish 802 and/or skin 116 during a cosmetic procedure session. LEDs 1550 could communicate with a source of power 1506 and computer 108. Alternatively and optionally and as shown in FIG. 15B, aperture 1512 could act as a partition preventing LEDs 1550 from being in direct contact with segment 150 of skin 116.

Referring now to FIG. 16, which is a simplified illustration of an image-based system for cosmetic procedures for skin in accordance with another example. In the example depicted in FIG. 16, computer 108, source of light energy 106, user interface 125 and display 122 could be housed inside or on a wall of applicator 1602 making applicator 1602 independent of external wire connections or a stationary base. It will be appreciated by those skilled in the art that a similar adjustment may be made mutatis mutandis for all of the examples described above, i.e., source of RF electrical energy 716 (FIGS. 7A and 7B) and source of power 1506 (FIG. 15) could also be housed in corresponding applicators 702 and 1502.

Also, there is provided a method including capturing an image of a segment of skin or blemish, analyzing and processing the captured image and extracting from the image information regarding the skin or blemish and determining specific optimal treatment doses of at least one of light energy and RF energy for one or more skin fractions within the segment of skin based on the extracted information.

Other forms of energy such as, for example, ultrasound energy employing, for example, an ultrasound array transducer could also be used mutatis mutandis alone or in combination with the energy forms described above to achieve the above described solutions.

It will be appreciated by persons skilled in the art that the present apparatus and method is not limited to what has been particularly shown and described hereinabove. Rather, the scope of the apparatus and method includes both combinations and sub-combinations of various features described hereinabove as well as modifications and variations thereof which would occur to a person skilled in the art upon reading the foregoing description and which are not in the prior art.

1-31. (canceled)

32. A system comprising:
an applicator including
at least one light energy emitter operative to emit light via an aperture;
at least one camera operative capture an image of skin via the aperture and communicate to a computer a captured image of a segment of skin including hair or a blemish; and
wherein the computer employs information extracted from the captured image of a segment of skin to determine specific optimal treatment light energy doses for at least one skin fractions within the segment of skin.

33. The system according to claim 32, wherein the specific optimal treatment light energy doses are based on at least one hair parameter selected from a group of parameters including number of hairs, density of hair, pigment of hair, length and thickness of hair and location of hair within the segment.

34. The system according to claim 32, wherein the light is a form of light energy applied in a light beam form including Intense Pulse Light (IPL), Light Emitting Diodes (LED) and a laser light selected from a group of lasers including gas lasers, solid-state lasers, fiber lasers, semiconductor lasers and dye lasers.

35. The system according to claim 34, wherein the computer is operative to automatically control the light dose applied to fractions of skin in accordance with a predetermined protocol defining preset light energy dose levels corresponding to an obtained set of image and/or skin parameters.

36. The system according to claim 32, wherein the system also comprises at least one sensor selected from a group of sensors including temperature sensors, impedance sensors and contact sensors.

37. The system according to claim 35, wherein the computer employs information extracted from at least one of the captured image of a segment of skin, manual input and sensors to formulate a cosmetic treatment protocol tailored to at least one parameter of at least one of hair, skin and blemish parameters and to determine specific optimal treatment light energy doses for one or more skin fractions within the segment of skin based on the at least one parameter.

38. The system according to claim 32, wherein the information extracted from the captured image includes at least one of skin and blemish parameters including skin type, location of segment of skin on body, level of pigmentation, type of blemish, temperature and/or impedance of segment of skin, level of skin hydration, blemish thickness, blemish shape and location within the segment of skin and/or blemish depth.

39. The system according to claim 32, wherein the system also comprises a display operative to display in real time images captured by the camera or images stored and retrieved from an image bank in the computer memory.

40. The system according to claim 32, further comprising:
a plurality of discrete voltage-applying elements; and
wherein the computer employs information extracted from the image to determine specific optimal treatment RF energy doses for one or more skin fractions within the segment of skin.

41. The system according to any claim 32, wherein the computer also employs information extracted from the image to determine specific light energy scanning patterns.

42. The system according to claim 32, wherein the captured image is displayed on a user interface touch-screen and allows a user, employing a finger or a stylus to at least one of outline an area or a segment within the displayed image to be treated or not treated, identify specific hairs, blemishes, scars or wrinkles to be treated and define a sequence and scanning pattern of application of light energy doses to two or more skin fractions within the segment of skin.

43. A system comprising:
an applicator including
at least one light energy emitter and an applicator computer; and
a remote image capturing and processing device, including
at least one camera operative to communicate to a remote computer an image of a segment of skin; and
wherein the second computer employs information extracted from the image of a segment of skin to determine specific optimal treatment doses of at least one of light energy and RF energy for at least one hair and/or hair containing skin fractions within the segment of skin and communicate the specific optimal treatment doses to the applicator computer.

44. The system according to claim 43, further comprising a plurality of discrete voltage-applying elements.

45. A system for personal skin treatment comprising:
a remote image capturing and processing device with at least one camera;
at least one light energy emitting hair treatment device; and
at least one communication link between the remote image capturing and processing device and the hair treatment device.

46. The system according to claim 45, wherein the system is operative to
automatically scan with the camera a large segment of skin;
communicate images of the scan captured by the camera to the computer; and wherein the computer is operative to:
display images of the scanned segment to be treated or not treated;
identify to the system specific blemishes, scars or wrinkles to be treated; and
define a sequence and scanning pattern of application of light energy doses to skin fractions within the scanned segment of skin.

47. The system according to claim 46, wherein the computer is also operative to display a list of blemishes to be treated and have a user confirm the listed blemishes to be treated; and
automatically activate application of appropriate light energy doses to confirmed blemishes to be treated.

48. The system according to claim 47, further comprising:
a plurality of discrete voltage-applying elements; and
wherein the computer employs information extracted from the image to determine specific optimal treatment RF energy doses for one or more skin fractions within the segment of skin.

49. The system according to claim 46, further comprising:
a plurality of discrete voltage-applying elements; and
wherein the computer employs information extracted from the image to determine specific optimal treatment RF energy doses for one or more skin fractions within the segment of skin.

50. The system according to claim 45, further comprising:
a plurality of discrete voltage-applying elements; and
wherein the computer employs information extracted from the image to determine specific optimal treatment RF energy doses for one or more skin fractions within the segment of skin.
